(19) United States
(12) Patent Application Publication
(10) Pub. No.: US 2005/0226934 A1
(43) Pub. Date: Oct. 13, 2005

(54) Inclusion complexes of active compounds in acrylate (co)polymers and methods for their production

(75) Inventors: Rina Goldshtein, Har Hebron (IL); Olga Sklyarsky, Rehovot (IL); Ilya Zelkind, Ofakim (IL); Michael Kopylov, Beer Sheva (IL); Boris Tulbovich, Ashkelon (IL); Vadim Goldshtein, Har Hebron (IL)

Correspondence Address: BROWDY AND NEIMARK, P.L.L.C.;624 NINTH STREET, NW, SUITE 300, WASHINGTON, DC, 20001-5303, US

(73) Assignee: SoluBest Ltd., Rehovot (IL)

(21) Appl. No.: 11/100,621

(22) Filed: Apr. 7, 2005

Related U.S. Application Data
(63) Continuation-in-part of application No. 10/952,380, filed on Sep. 29, 2004.
(63) Continuation-in-part of application No. 10/256,023, filed on Sep. 26, 2002.
Application No. 10/256,023 is a continuation-in-part of application No. 09/966,847, filed on Sep. 28, 2001, now Pat. No. 6,878,693.
(60) Provisional application No. 60/507,623, filed on Sep. 30, 2003.

(51) Int. Cl. A61K 9/14

(52) U.S. Cl. 424/487

(57) ABSTRACT

The present invention provides a hydrophilic inclusion complex consisting essentially of nanosized particles of a non-crystalline active compound wrapped by an amphiphilic polymer consisting of a homopolymer of acrylic acid or methacrylic acid or a copolymer of acrylic acid or methacrylic acid, or both, with one or more comonomers. The invention further provides hydrophilic dispersions comprising nanoparticles of said inclusion complexes and stable compositions comprising them.

### CROSS-REFERENCE TO RELATED APPLICATIONS

The present application is a continuation-in-part of application Ser. No. No. 10/952,380, filed Sep. 29, 2004, which is a non-provisional of the Provisional Application No. 60/507,623, filed Sep. 30, 2003 and a continuation-in-part of application Ser. No. 10/256,023, filed Sep. 26, 2002, which is a continuation-in-part of application Ser. No. 09/966,847, filed Sep. 28, 2001, the entire contents of each and all these applications being hereby incorporated by reference herein in their entirety as if fully disclosed herein.

### FIELD OF THE INVENTION

The present invention is in the field of nanoparticles. More particularly, the invention relates to soluble nanosized particles consisting of inclusion complexes of an active compound wrapped within poly(meth)acrylate amphiphilic polymers, and methods of producing said nanoparticles.

### BACKGROUND OF THE INVENTION

Two formidable barriers to effective drug delivery and hence to disease treatment, are solubility and stability. To be absorbed in the human body, a compound has to be soluble in both water and fats (lipids). Solubility in water is, however, often associated with poor fat solubility and vice-versa.

Over one third of drugs listed in the U.S. Pharmacopoeia and about 50% of new chemical entities (NCEs) are insoluble or poorly insoluble in water. Over 40% of drug molecules and drug compounds are insoluble in the human body. In spite of this, lipophilic drug substances having low water solubility are a growing drug class having increasing applicability in a variety of therapeutic areas and for a variety of pathologies.

Solubility and stability issues are major formulation obstacles hindering the development of therapeutic agents. Aqueous solubility is a necessary but frequently elusive property for formulations of the complex organic structures found in pharmaceuticals. Traditional formulation systems for very insoluble drugs have involved a combination of organic solvents, surfactants and extreme pH conditions. These formulations are often irritating to the patient and may cause adverse reactions.

The size of the drug molecules also plays a major role in their solubility and stability as well as bioavailability. Bioavailability refers to the degree to which a drug becomes available to the target tissue or any alternative in vivo target (i.e., receptors, tumors, etc.) after being administered to the body. Poor bioavailability is a significant problem encountered in the development of pharmaceutical compositions, particularly those containing an active ingredient that is poorly soluble in water. Poorly water-soluble drugs tend to be eliminated from the gastrointestinal tract before being absorbed into the circulation. It is known that the rate of dissolution of a particulate drug can increase with increasing surface area, that is, decreasing particle size

Recently, there has been an explosion of interest in nanotechnology, the manipulation on the nanoscale. Nanotechnology is not an entirely new field: colloidal sols and supported platinum catalysts are nanoparticles. Nevertheless, the recent interest in the nanoscale has produced, among numerous other things, materials used for and in drug delivery. Nanoparticles are generally considered to be solids whose diameter varies between 1-1000 nm.

Although a number of solubilization technologies do exist, such as liposomes, cylcodextrins, microencapuslation, and dendrimers, each of these technologies has a number of significant disadvantages.

Liposomes, as drug carriers, have several potential advantages, including the ability to carry a significant amount of drug, relative ease of preparation, and low toxicity if natural lipids are used. However, common problems encountered with liposomes include: low stability, short shelf-life, poor tissue specificity, and toxicity with non-native lipids. Additionally, the uptake by phagocytic cells reduces circulation times. Furthermore, preparing liposome formulations that exhibit narrow size distribution has been a formidable challenge under demanding conditions, as well as a costly one. Also, membrane clogging often results during the production of larger volumes required for pharmaceutical production of a particular drug.

Cyclodextrins are crystalline, water-soluble, cyclic, non-reducing oligo-saccharides built from six, seven, or eight glucopyranose units, referred to as alpha, beta and gamma cyclodextrin, respectively, which have long been known as products that are capable of forming inclusion complexes. The cyclodextrin structure provides a molecule shaped like a segment of a hollow cone with an exterior hydrophilic surface and interior hydrophobic cavity. The hydrophilic surface generates good water solubility for the cyclodextrin and the hydrophobic cavity provides a favorable environment in which to enclose, envelope or entrap the drug molecule. This association isolates the drug from the aqueous solvent and may increase the drug's water solubility and stability.

For a long time, most cyclodextrins had been no more than scientific curiosities due to their limited availability and high price, but lately cyclodextrins and their chemically modified derivatives became available commercially, generating a new technology of packing on the molecular level. Cyclodextrins are, however, fraught with disadvantages including limited space available for the active molecule to be entrapped inside the core, lack of pure stability of the complex, limited availability in the marketplace, and high price.

Microencapsulation is a process by which tiny parcels of a gas, liquid, or solid active ingredient (“core material”) are packaged within a second material for the purpose of shielding the active ingredient from the surrounding environment. These capsules, which range in size from one micron (one-thousandth of a millimeter) to approximately seven millimeters, release their contents at a later time by means appropriate to the application.

There are four typical mechanisms by which the core material is released from a microcapsule: (1) mechanical rupture of the capsule wall, (2) dissolution of the wall, (3) melting of the wall, and (4) diffusion through the wall. Less common release mechanisms include ablation (slow erosion of the shell) and biodegradation.

Microencapsulation covers several technologies, where a certain material is coated to obtain a micro-package of the active compound. The coating is performed to stabilize the material, for taste masking, preparing free flowing material of otherwise clogging agents etc. and many other purposes. This technology has been successfully applied in the feed additive industry and to agriculture. The relatively high production cost needed for many of the formulations is, however, a significant disadvantage.

In the cases of nanoencapsulation and nanoparticles (which are advantageously shaped as spheres and, hence, nanospheres), two types of systems having different inner structures are possible: (i) a matrix-type system composed of an entanglement of oligomer or polymer units, defined as nanoparticles or nanospheres, and (ii) a reservoir-type system, consisting of an oily core surrounded by a polymer wall, defined as a nanocapsule.

Depending upon the nature of the materials used to prepare the nanospheres, the following classification exists: (a) amphiphilic macromolecules that undergo a cross-linking reaction during preparation of the nanospheres; (b) monomers that polymerize during preparation of the nanoparticles; and (c) hydrophobic polymers, which are initially dissolved in organic solvents and then precipitated under controlled conditions to produce nanoparticles.

Problems associated with the use of polymers in micro- and nanoencapsulation include the use of toxic emulgators in emulsions or dispersions, polymerization or the application of high shear forces during emulsification process, insufficient biocompatibility and biodegradability, balance of hydrophilic and hydrophobic moieties, etc. These characteristics lead to insufficient drug release.

Dendrimers are a class of polymers distinguished by their highly branched, tree-like structures. They are synthesized in an iterative fashion from ABn monomers, with each iteration adding a layer or “generation” to the growing polymer. Dendrimers of up to ten generations have been synthesized with molecular weights in excess of 106 kDa. One important feature of dendrimeric polymers is their narrow molecular weight distributions. Indeed, depending on the synthetic strategy used, dendrimers with molecular weights in excess of 20 kDa can be made as single compounds.

Dendrimers, like liposomes, display the property of encapsulation, and are able to sequester molecules within the interior spaces. Because they are single molecules, not assemblies, drug-dendrimer complexes are expected to be significantly more stable than liposomal drugs. Dendrimers are thus considered as one of the most promising vehicles for drug delivery systems. However, the dendrimer technology is still in the research stage, and it is speculated that it will take years before it is applied in the industry as an efficient drug delivery system.

### SUMMARY OF THE INVENTION

Lipophilic and hydrophilic compounds that are solubilized in the form of nanosized particles, or nanoparticles, can be used in pharmaceutical products, in the production of food additives, cosmetics, and agriculture, as well as in pet foods and veterinary products, amongst other uses.

The present invention provides nanoparticles and methods for the production of soluble nanoparticles and, in particular, inclusion complexes of water-insoluble lipophilic and water-soluble hydrophilic organic materials wrapped by amphiphilic polymers, more specifically by a homopolymer of acrylic acid or methacrylic acid or by a copolymer of acrylic acid or methacrylic acid, or both.

The hydrophilic inclusion complex of the present invention consists essentially of nanosized particles of an active compound and an amphiphilic polymer which wraps said active compound such that non-valent bonds are formed between said active compound and said amphiphilic polymer in said inclusion complex, wherein said amphiphilic polymer is a homopolymer of acrylic acid or methacrylic acid or a copolymer of acrylic acid or methacrylic acid, or both.

The present invention further relates to hydrophilic dispersions comprising nanoparticles of the said inclusion complexes, to methods for their preparation and to stable pharmaceutical and pesticidal compositions comprising said dispersions.

### DETAILED DESCRIPTION OF THE INVENTION

The present invention provides nanoparticles and methods for the production of soluble nanoparticles and, in particular, hydrophilic dispersions of nanoparticles of inclusion complexes of an active compound in certain amphiphilic polymers.

The soluble nanoparticles, referred to herein sometimes as “solu-nanoparticles” or “solumers”, are differentiated by the use of water-soluble amphiphilic polymers that are capable of producing molecular complexes with lipophilic and hydrophilic active compounds or molecules, particularly pharmaceutical drugs. The solu-nanoparticles formed in accordance with the present invention render water-insoluble active compounds soluble in water and readily bioavailable in the human body.

As used herein, the term “inclusion complex” refers to a complex in which one component—the amphiphilic polymer (the “host), forms a cavity in which molecular entities of a second chemical species—the active compound (the “guest”), are located. Thus, in accordance with the present invention, inclusion complexes are provided in which the host is the amphiphilic polymer and the guest is the active compound molecules wrapped and fixated or secured within the cavity or space formed by said amphiphilic polymer host.

In accordance with the present invention, the inclusion complexes contain the active compound molecules, which interact with the polymer by non-valent interactions and form a polymer-active compound as a distinct molecular entity. A significant advantage and unique feature of the inclusion complex of the present invention is that no new chemical bonds are formed and no existing bonds are destroyed during the formation of the inclusion complex (very important for pharmaceutical drugs). The particles comprising the inclusion complexes are nanosized and no change occurs in the active compound molecule itself, when it is enveloped, or advantageously wrapped, by the polymer.

Another important characteristic of the inclusion complex of the invention is that the active compound may be presented in a non-crystalline state. As used herein, the term “non-crystalline” state is intended to include both disordered crystalline and, preferably, amorphous state. Thus, in preferred embodiments, the active compound is in amorphous form. It is known in the art that the amorphous state is preferred for drug delivery as it may indeed enhance bioavailability.

The creation of the complex does not involve the formation of any valent bonds (which may change the characteristics or properties of the active compound). As used herein, the term “non-valent” is intended to refer to non-covalent, non-ionic and non-semi-polar bonds and/or interactions including weak, non-covalent bonds and/or interactions such as electrostatic forces, van der Waals forces and hydrogen bonds formed during the creation of the inclusion complex. The formation of non-valent bonds preserves the structure and properties of the active compound.

The solunanoparticles of the invention remain stable for long periods of time, may be manufactured at a low cost, and may improve the overall bioavailability of the active compound.

In one aspect, the present invention relates to a hydrophilic inclusion inclusion complex consisting essentially of nanosized particles of an active compound and an amphiphilic polymer which wraps said active compound such that non-valent bonds are formed between said active compound and said amphiphilic polymer in said inclusion complex, wherein said amphiphilic polymer is a homopolymer of acrylic acid or methacrylic acid, or a copolymer of acrylic acid, methacrylic acid, or both.

As used herein, the term “copolymer of acrylic acid, methacrylic acid, or both” refers to: (i) a copolymer of acrylic acid with one or two different comonomers; (ii) a copolymer of methacrylic acid with one or two different comonomers; and (iii) a copolymer of acrylic acid with methacrylic acid and another comonomer.

In one embodiment, the amphiphilic polymer is a homopolymer of acrylic acid or methacrylic acid. In another embodiment, the amphiphilic polymer is a copolymer of acrylic acid or methacrylic acid with one or two monomers selected from the group consisting of acrylamide, methacrylamide, an alkyl acrylate, an alkyl methacrylate, acrylonitrile, ethyleneimine, vinyl acetate, styrene, maleic anhydride and vinyl pyrrolidone. In a further embodiment, the amphiphilic polymer is a copolymer of acrylic acid, methacrylic acid and a third monomer selected from the group consisting of acrylamide, methacrylamide, an alkyl acrylate, an alkyl methacrylate, acrylonitrile, ethyleneimine, vinyl acetate, styrene, maleic anhydride and vinyl pyrrolidone. Polar monomers such as acrylic acid, methacrylic acid and acrylamide impart mechanical stability to the polymer and contribute to the hydrophilicity of the polymer.

In some preferred embodiments, the amphiphilic polymer is a copolymer of acrylic acid or methacrylic acid with an alkyl acrylate or an alkyl methacrylate. In other preferred embodiments, the amphiphilic polymer is a copolymer of acrylic acid, methacrylic acid and an alkyl acrylate or an alkyl methacrylate. The alkyl radical of the alkyl acrylate or alkyl methacrylate may be a straight, branched or cyclic (C₁-C₁₂)alkyl, preferably (C₁-C₈)alkyl, more preferably (C₁-C₄)alkyl, optionally substituted by a radical selected from the group consisting of OH, —CONH₂, —NH₂, —COOH, —SO₃H, and —PO₃H₂.

Examples of straight, branched or cyclic (C₁-C₁₂)alkyl acrylate and methacrylate include, without being limited to, methyl acrylate, ethyl acrylate, butyl acrylate, n-octyl acrylate, 2-ethylhexyl acrylate and cyclohexyl methacrylate. The alkyl may preferably be substituted by hydroxyl and examples of such esters include, without limitation, 2-hydroxyethyl acrylate, 2-hydroxyethyl methacrylate, and 2-hydroxypropyl methacrylate.

In some preferred embodiments, the amphiphilic polymer consists of copolymers with various ratios of acrylic acid and butyl acrylate. In other preferred embodiments, the amphiphilic polymer consists of copolymers with various ratios of acrylic acid, methacrylic acid and butyl acrylate. In further preferred embodiments, the amphiphilic polymer consists of copolymers with various ratios of acrylic acid and 2-hydroxyethyl methacrylate.

The nanoparticles of the present invention comprise the insoluble or soluble active compound or core wrapped within a water-soluble amphiphilic copolymer. As described in the parent U.S. applications Ser. No. 10/256,023 and Ser. No. 09/966,847, hereby incorporated by reference in their entirety, a variety of different polymers can be used for any of the selected lipophilic or hydrophilic active compound, and the polymer, or groups of polymers, is selected according to an algorithm that takes into account various physical properties of both the active lipophilic or hydrophilic compound and the interaction of this compound within the resulting active compound /polymer nanosoluparticle.

One important parameter in the choice of the polymer or polymers is the HLB, i.e., the measure of the molecular balance of the hydrophilic and lipophilic portions of the compound. Within the HLB International Scale of 0-20, lipophilic molecules have a HLB of less than 6, and hydrophilic molecules have a HLB of more than 6. Thus, according to the present invention, the HLB of the polymer is selected in such a way that, after combining to it the active compound, the total resulting HLB value of the complex will be greater than 8, rendering the complex water-soluble.

The active compound may be selected from the group consisting of pharmaceutical compounds, food additives, cosmetics, pesticides and pet foods.

In some preferred embodiments, the active compound is selected from the group consisting of vitamins, antibiotics and hormones. In another embodiment, the active compound is a pharmaceutical compound.

In one embodiment of the present invention, the active compound is an azole compound. Azole compounds play a key role as antifungals in agriculture and in human mycoses and as nonsteroidal antiestrogens in the treatment of estrogen-responsive breast tumors in postmenopausal women. This broad use of azoles is based on their inhibition of certain pathways of steroidogenesis by high-affinity binding to the enzymes sterol 14-demethylase and aromatase. Azole fungicides show a broad antifungal activity and are used either to prevent fungal infections or to cure an infection. Therefore, they are important tools in integrated agricultural production. According to their chemical structure, azole compounds are classified into triazoles and imidazoles; however, their antifungal activity is due to the same molecular mechanism. Azole fungicides are broadly used in agriculture and in human and veterinary antimycotic therapies.

In accordance with the present invention, an “azole compound” refers to imidazole and triazole compounds for human or veterinary application or for use in the agriculture.

In one preferred embodiment, the azole compound is selected from azole fungicides for human application in many different antimycotic formulations including, but not limited to, the triazoles terconazole, itraconazole, and fluconazole, and the imidazoles clotrimazole, miconazole, econazole, ketoconazole, tioconazole, isoconazole, oxiconazole, and fenticonazole.

In one more preferred embodiment, the azole compound for human application is itraconazole, an azole medicine used to treat fungal infections. It is effective against a broad spectrum of fungi including dermatophytes (tinea infections), yeasts such as candida and malassezia infections, and systemic fungal infections such as histoplasma, aspergillus, coccidiodomycosis, chromo-blastomycosis. Itraconazole is available as 100 mg capsules under the trademark Sporanox™ (Janssen-Cilag). It is a white to slightly yellowish powder. It is lipophilic, insoluble in water, very slightly soluble in alcohols, and freely soluble in dichloromethane. Sporanox contains 100 mg of itraconazole coated on sugar spheres. According to the present invention, the inclusion complex contains itraconazole wrapped by a polymer selected from the group consisting of polyacrylic acid, a copolymer of acrylic acid with butyl acrylate, a copolymer of acrylic acid, methacrylic acid and butyl acrylate, and a copolymer of acrylic acid and 2-(hydroxyethyl)methacrylate.

In another embodiment, the azole compound is selected from azoles that act as nonsteroidal antiestrogens and can be used in the treatment of estrogen-responsive breast tumors in postmenopausal women, including, but not limited to letrozole, anastrozole, vorozole, and fadrozole.

In another embodiment, the azole compound is an azole fungicide useful in the agriculture including, but not limited to, the triazoles bitertanol, cyproconazole, difenoconazole, epoxiconazole, fluquinconazole, flusilazole, flutriafol, hexaconazole, metconazole, myclobutanil, penconazole, propiconazole, tebuconazole, triadimefon, triadimenol, and triticonazole, and the imidazoles imazalil, prochloraz, and triflumizole. In still another embodiment, the azole compound is a nonfungicidal azole for use in the agriculture such as the triazoles azocyclotin used as an acaricide, paclobutrazole as a growth regulator, carfentrazone as a herbicide, and isazophos as an insecticide, and the imidazole metazachlor used as herbicide.

In another preferred embodiment, the azole compound is tebuconazole, a triazole systemic fungicide with protective, curative and eradicant action, that inhibits ergosterol biosynthesis. It is lipophilic, insoluble in water and acetone, and freely soluble in dichloroethane, toluene and isopropanol. According to the present invention, the inclusion complex contains tebuconazole wrapped by a copolymer of acrylic acid and butyl acrylate.

In another aspect, the present invention provides a hydrophilic dispersion comprising nanoparticles of inclusion complexes as defined above. Thus, the present invention provides a hydrophilic dispersion of water-soluble and stable nanoparticles of inclusion complexes consisting essentially of nanosized particles of an active compound and an amphiphilic homopolymer of acrylic acid or methacrylic acid or copolymer of acrylic acid and/or methacrylic acid which wraps said active compound such that non-valent bonds are formed between said active compound and said amphiphilic polymer in said inclusion complex.

The dispersions of the invention are stable. Stability of the nanoparticles and of the inclusion complexes has more than one meaning. The nanoparticles should be stable as part of a nanocomplex over time, while remaining in the dispersion media. The nanodispersions are stable over time without separation of phases. Furthermore, the non-crystalline or amorphous state should be also retained over time.

It is worth noting that in the process used in the present invention, the components of the system do not result in micelles nor do they form classical dispersion systems. The technology of the present invention causes the following:
(i) after dispersion of the active compound to nano-sized particles and fixation by the polymer to form an inclusion complex, enhanced solubility in physiological fluids, in vivo improved absorption, and improved biological activity, as well as transmission to a stable non-crystalline, preferably amorphous, state, are achieved;
(ii) the otherwise crystalline biologically-active compound becomes non-crystalline, e.g., amorphous, and thus exhibits improved biological activity.

In most preferred embodiments of the present invention, not less than 80% of the nanoparticles in the nanodispersion are within the size range, when the size deviation is not greater than 20%, and the particle size is within the nano range, namely less than 1000 nm, more preferably 100 nm or less.

In an advantageous and preferred embodiment of the invention, the copolymer molecule wraps the active compound via non-valent interactions. between the (co)polymer and the active compound in the inclusion complex such that the non-valent interactions fixate the active compound within the (co)polymer thus reducing its molecular mobility. The formation of any valent bonds could change the characteristics or properties of the active compound. The formation of non-valent bonds preserves the structure and properties of the active compound, which is particularly important when the active compound is a pharmaceutical.

The hydrophilic dispersions comprising the nanoparticles of the inclusion complexes of the invention can be prepared by the method described in the parent U.S. applications Ser. No. 10/952,380, Ser. No. 10/256,023 and Ser. No. 09/966,847, hereby incorporated by reference in their entirety, whereby the polymerization reaction is first carried out in an aqueous solution and a molecular solution of the active compound in an organic solvent is added to the polymer solution. Evaporation of the organic solvent leads to the desired dispersion. This process is herein designated “two-step process”.

The present invention thus provides a process for preparation of a hydrophilic dispersion comprising nanoparticles of inclusion complexes of an active compound and an amphiphilic polymer which wraps the active compound such that non-valent bonds are formed between said active compound and said amphiphilic polymer, wherein said amphiphilic polymer is a homopolymer of acrylic acid or methacrylic acid or a copolymer of acrylic acid or methacrylic acid, or both, the process comprising the steps of:
(i) preparing the amphiphilic homopolymer or copolymer of acrylic acid and/or methacrylic acid by reaction of the monomer(s) in water;
(ii) preparing a molecular solution of the active compound in an organic solvent;
(iii) dripping the cold solution of the active compound (ii) into the heated water homopolymer or copolymer solution (i) and heating at a temperature 5 to 10° C. above the boiling point of the organic solvent, under constant mixing; and
(iv) removing the organic solvent,
thus obtaining the hydrophilic dispersion comprising nanoparticles of inclusion complexes of said active compound wrapped within said amphiphilic homopolymer of acrylic acid or methacrylic acid or copolymer of acrylic acid or methacrylic acid, or both.

The dispersions of the invention can also be obtained by a method in which the polymerization process and formation of the nanoparticles occur simultaneously in the same reaction flask. This method of concurrent polymerization and solumerization is herein designated “one-step process”.

In one embodiment, the one-step process is carried out with an organic solvent. Thus, the present invention further provides a one-step process for preparation of a hydrophilic dispersion comprising nanoparticles of inclusion complexes of an active compound and an amphiphilic polymer which wraps the active compound such that non-valent bonds are formed between said active compound and said amphiphilic polymer in said inclusion complex, wherein said amphiphilic polymer is a homopolymer of acrylic acid or methacrylic acid or a copolymer of acrylic acid or methacrylic acid, or both, the process comprising the steps of:
(i) preparing a solution of the monomers in an organic solvent;
(ii) preparing a molecular solution of the active compound in a portion of the organic solution of (i);
(iii) dripping the remaining portion of solution (i) into a heated aqueous medium containing a polymerization initiator;
(iv) dripping the solution (ii) of the active compound into the heated aqueous initiator medium with the added monomers solution (i) and heating at 70-90° C., under constant mixing; and
(v) removing the organic solvent,
thus obtaining the hydrophilic dispersion comprising nanoparticles of inclusion complexes of said active compound wrapped within said amphiphilic homopolymer of acrylic acid or methacrylic acid or copolymer of acrylic acid or methacrylic acid, or both.

In another embodiment, the one-step process is carried out without an organic solvent. Thus, the present invention further provides a one-step process for preparation of a hydrophilic dispersion comprising nanoparticles of inclusion complexes of an active compound and an amphiphilic polymer which wraps the active compound such that non-valent bonds are formed between said active compound and said amphiphilic polymer in said inclusion complex, wherein said amphiphilic polymer is a homopolymer of acrylic acid or methacrylic acid or a copolymer of acrylic acid or methacrylic acid, or both, the process comprising the steps of:
(ii) providing acrylic acid, acrylic acid or a mixture of monomers in liquid form;
(ii) preparing a molecular solution of the active compound in a portion of the monomer or the monomer mixture of (i);
(iii) dripping the remaining portion of the monomer or the monomer mixture of (i) into a heated aqueous medium containing a polymerization initiator; and
(iv) dripping the solution (ii) of the active compound into the heated aqueous initiator medium with the added monomers solution of (iii) and heating at 70-90° C., under constant mixing;
thus obtaining the hydrophilic dispersion comprising nanoparticles of inclusion complexes of said active compound wrapped within said amphiphilic homopolymer of acrylic acid or methacrylic acid or copolymer of acrylic acid or methacrylic acid, or both.

The copolymers of the invention are obtained from acrylic acid or methacrylic acid monomers, or both, and the additional comonomer(s) by free-radical polymerization with or without addition of branching agents.

According to the invention, a solution of acrylic acid or methacrylic acid in a solvent is dripped into an aqueous initiator medium under continuous stirring and heating. As solvent, water, acetone, methyl acetate or mixture thereof can be used to dissolve the acrylic acid or methacrylic acid monomers and the comonomers. The amount of solvent, based on the entirety of acrylic/methacrylic acid and comonomers is generally from 0 to 150% by weight. When an organic solvent is used, it is distilled off from the reaction mixture during the polymerization. The amount of water in the reaction mixture during the polymerization remains practically constant. The resulting polymer solution is about 10-40% by weight.

In the batch operation, the monomers may be fed to the reactor during from 1 to 2 hours. On completion of the dripping, the polymerization of the reaction mixture is usually continued for from 1 to 2 hours. Any residues of organic solvent present may be distilled out from the polymerization mixture at this time. The temperature of reactions carrying out is varied from 70° C. up to 95° C.

The polymerization initiators useful for the purpose of the invention may be any of the known water-soluble peroxo initiators. Particularly preferred polymerization initiators are hydrogen peroxide and the peroxodisulfates of sodium, potassium and of ammonium. The amounts of initiator are usually from 0.1 to 20% by weight, preferably from 3 to 7.5% by weight, based on the monomers to be polymerized.

The preferred comonomers in the preparation of the acrylic/methacrylic acid copolymers are butyl acrylate and 2-(hydroxyethyl)-methacrylate, but any other comonomer as defined hereinabove may be used according to the invention. The amount of comonomer, based on acrylic/methacrylic acid, is generally from 1 to 200% by weight, more preferably from 10 to 12.5%.

A branching agent may be added into the aqueous initiator medium such as, but not limited to, polyhydroxy agents such as triethanolamine, glycerol, trimethylolpropane, 1,2,4-butanetriol, ethylene glycol, 1,3-propanediol, 1,4-butanediol, 1,6-hexanediol, pentaerythritol, sorbitol, polyvinyl alcohol and mixtures thereof. In the preparation of the acrylic/methacrylic acid copolymers, the amount of branching agent, based on the entirety of acrylic/methacrylic acid and comonomers, may be from 0.03 to 3% by weight, more preferably from 0.3 to 1%.

It is shown herein that the polyacrylate polymers composed of homopolymers of acrylic acid or methacrylic acid and of copolymers of acrylic acid or methacrylic acid, or both, with various comonomers provide a distinct polymer platform for use in applying the herein designated Solumer technology to produce Solumer inclusion complexes for use in pharmaceutical and other formulations, depending on the active compound that is solumerized by the acrylate (co)polymer.

The aqueous nanodispersions of the invention can be lyophilized and then mixed with pharmaceutically acceptable carriers to provide stable pharmaceutical compositions.

The pharmaceutically acceptable carriers or excipients are adapted to the active compound and the type of formulation and can be chosen from standard excipients as well-known in the art, for example, as described in Remington: The Science and Practice of Pharmacy (Formerly Remington's Pharmaceutical Sciences) 19th ed., 1995.

Thus, in another aspect, the present invention provides stable pharmaceutical compositions comprising pharmaceutically acceptable carriers and a nano-dispersion of the invention. The compositions are preferably for oral administration, and may be in liquid or solid form.

In a preferred embodiment, the invention relates to stable pharmaceutical composition for treatment of fungal infections comprising a nanodispersion of water-soluble nanoparticles of inclusion complexes wherein the active compound is an azole fungicide and the amphiphilic polymer is a copolymer of acrylic acid or methacrylic acid. Most preferably, the azole fungicide is itraconazole and the amphiphilic polymer is polyacrylic acid or a copolymer of acrylic acid with butyl acrylate or with 2-hydroxyethyl methacrylate in different proportions.

In another aspect, the present invention provides a stable pesticidal composition for treatment of fungal infections in agricultural crop comprising an agriculturally acceptable carrier and nanodispersion of water-soluble nanoparticles of inclusion complexes wherein the active compound is an azole fungicide and the amphiphilic polymer is a copolymer of acrylic acid or methacrylic acid. Most preferably, the azole fungicide is tebuconazole and the amphiphilic polymer is a copolymer of acrylic acid with butyl acrylate in different proportions.

The invention will now be illustrated by the following non-limiting examples.

### EXAMPLES

### Methods

### (i) Preparation of Polymers

Radical polymerization is carried out in a three-neck round-bottom flask provided with a drop funnel, a high-speed homogenizer, and a vapor condenser. Aqueous, acetone or methyl acetate solution of monomer(s) is added dropwise into an aqueous initiator medium under continuous stirring (6500-20000 rpm) and heating (70° C.-95° C.). On completion of the dripping, the reaction mixture is heated and stirred for yet 1-2 hours. The organic solvent is distilled off from the reaction mixture during the polymerization.

### (ii) Preparation of Dispersions of Nanoparticles of Inclusion Complexes of Active Compounds (Solumerization of Active Compounds)

The aqueous polymer solution of (i) is placed into a in a three-neck round-bottom flask provided with a drop funnel, a high-speed homogenizer, and a vapor condenser. A solution of an active compound in methylene chloride, or acetone, or methyl acetate, or a mixture thereof, is added dropwise into the reaction flask under continuous stirring (6500 rpm and more) and heating (60° C.-75° C.). On completion of the dripping, the obtained mixture is heated and stirred for yet 0.5 hours for evaporation of residual organic solvent, thus obtaining the dispersion comprising nanoparticles of inclusion complexes of the active compound wrapped by the polymer.

### (iii) Concurrent Polymerization and Solumerization (i.e., Preparation of Dispersions of Nanoparticles of Inclusion Complexes of Active Compound)

The processes of polymerization and solumerization, namely, formation of hydrophilic dispersions of nanoparticles of inclusion complexes of an active compound wrapped by the polymer, are carried out simultaneously in a three-neck round-bottom flask provided with a drop funnel, a high-speed homogenizer, and a vapor condenser. An organic phase is created by dissolving the monomer(s) in an organic solvent and adding part of the organic solution dropwise to a solution of an initiator in water under continuous stirring (6500-20000 rpm) and heating (75° C.-85° C.), followed by addition of the active compound dissolved in the remaining part of the organic phase. On completion of the dripping, the reaction mixture is heated and stirred for yet 1-2 hours. The organic solvent is distilled off from the reaction mixture during the process.

### (iv) Chemical and Physicochemical Analysis

The resulting polymer solutions were tested for acidity, viscosity and turbidity, and the solumers (dispersions of nanoparticles) were tested for content of active compound, turbidity, particles size and amorphousity, using the following techniques:
(a) The content of active compound was measured by HPLC.
(b) Particles sizes were measured by Zetasizer Nano-ZS, Malvern Instruments.
(c) Amorphous structures of active materials were studied by at least one of three techniques: (i) X-Ray diffractometry, used to identify crystalline compounds carried out with a theta-theta powder diffractometer Thermo ARL (formerly Scintag, Inc.) equipped with liquid nitrogen cooled Ge solid-state detector; (ii) Differential Scanning Calorimetry (DSC). This technique, by measuring the heat absorbed or given off by a sample as it is heated or cooled under a controlled temperature and atmosphere, is able to record changes in specific heat capacity and latent heat that indicate changes in amorphous and crystalline structures. DSC tests were carried out with TA Instruments module 2010 and System Controller 2100 at a scan rate of 10 deg/min from −50° C. to 200° C.; (iii) Fourier Transform Infrared (FTIR) spectroscopy, a powerful analytical tool for characterizing and identifying organic molecules.
(d) The viscosity of polymers and solumers was measured with a viscometer Visco Star Plus (Fungilab S A, Barcelona, Spain).

### Example 1

### Preparation of Polymers Solutions

### i. Acrylic acid-butyl acrylate copolymer 33% solution (Copolymer A)

For the preparation of copolymer A solution, 5 g of ammonium peroxodisulfate in 175 ml of distilled water were placed into a reaction flask. A mixture consisting of 66 ml of acrylic acid and 9 ml of butyl acrylate was added dropwise into the reaction flask, under continuous stirring (10500 rpm) and heating (86° C.), during 200 min. After the dripping, the reaction mixture was heated and stirred for yet 2 hours.

The resulting acrylic acid-butyl acrylate copolymer 33.3% solution is a yellowish opalescent liquid with viscosity 195 cps (25° C., Sp L2, 100 rpm), pH 1.18.

### ii. Acrylic acid-butyl acrylate copolymer 30% solution (Copolymer B)

For the preparation of copolymer B solution, 5.6 g of ammonium peroxodisulfate in 160 ml of distilled water were placed into a reaction flask. A mixture consisting of 57.6 ml of acrylic acid, 2.4 ml of butyl acrylate and 60 ml of methyl acetate was added dropwise into the reaction flask, under continuous stirring (8500 rpm) and heating (80° C.), during 130 min. After the dripping, the reaction mixture was heated and stirred for yet 1 hour.

The resulting acrylic acid-butyl acrylate copolymer 30% solution was a transparent colorless liquid with viscosity 107 cps (25° C., Sp L2, 200 rpm), pH 1.3.

### iii. Acrylic acid-2-hydroxyethyl methacrylate copolymer solution (Copolymer C)

For the preparation of copolymer C solution, 6 g of ammonium peroxodisulfate dissolved in 90 ml of distilled water were placed into a reaction flask. A mixture consisting of 54 ml of acrylic acid, 6 ml of 2-hydroxyethyl methacrylate, and 70 ml of distilled water was added dropwise into the reaction flask, under continuous stirring (11500 rpm) and heating (90° C.) of the solution, during 55 minutes. On completion of the dripping, the reaction mixture was heated and stirred for yet 1 hour.

The resulting acrylic acid-2-hydroxyethyl methacrylate copolymer 30% solution was a clear colorless liquid with viscosity 59 cps (25° C., Sp L2, 200 rpm).

### iv. Acrylic acid-methacrylic acid-butyl acrylate copolymer solution (Copolymer D)

For the preparation of copolymer D solution, 6 g of ammonium peroxodisulfate dissolved in 90 ml of distilled water were placed into a reaction flask. A mixture consisting of 52.8 ml of acrylic acid, 3.6 ml of methacrylic acid, 3.6 ml of butyl acrylate, 70 ml of distilled water, and 20 ml of methyl acetate was added dropwise into the reaction flask, under continuous stirring (10500 rpm) and heating (90° C.) of the solution, during 50 minutes. On completion of the dripping, the reaction mixture was heated and stirred for yet 2 hours.

The resulting acrylic acid-methacrylic acid-butyl acrylate copolymer 30% solution was a clear yellowish liquid with viscosity 61 cps (25° C., SpL2, 200 rpm).

### v. Acrylic acid polymer 33% solution (Polymer E)

For the preparation of polymer E solution, 5 g of ammonium peroxodisulfate in 175 ml distilled water were placed into a reaction flask. Then, 75 ml of acrylic acid were added dropwise into the reaction flask, under continuous stirring (10500 rpm) and heating (85° C.) of the solution, during 140 minutes. On completion of the dripping, the reaction mixture was heated and stirred for yet another 65 min, and cooled after that for 85 min to 45° C.

The resulting acrylic acid polymer 33% solution was a transparent colorless liquid with viscosity 280 cps (SpL2, 60 rpm), pH=1.24.

### Example 2

### Preparation of Dispersions of Nanoparticles of Inclusion Complexes of Itraconazole Wrapped in Copolymer A (Solumer A)

For the preparation of the itraconazole Solumer A, 200 ml of Copolymer A solution prepared in Example 1 (i) was placed into a reaction flask. 290 ml of a 1% itraconazole solution in methylene chloride were dripped into the reaction flask, under continuous stirring (10500 rpm) and heating (62° C.), for 12 hours. With this operation completed, the reaction mixture was heated and stirred for yet 1 hour.

The resulting dispersion comprising the itraconazole Solumer A was a clear yellow liquid with average particles size of 284.7 nm at 37° C. and itraconazole exhibited an amorphous structure as measured by DSC, X-Ray and FTIR. Viscosity was 270 cps (SpL2, 100 rpm). The content of itraconazole was 12.6 mg/ml (84% from the amount initially introduced) measured two months later by HPLC.

### Example 3

### Preparation of Dispersions of Nanoparticles of Inclusion Complexes of Itraconazole Wrapped in Copolymer B (Solumer B)

For the preparation of the itraconazole Solumer B, 60 ml of Copolymer B solution prepared in Example 1(ii) was placed into a reaction flask. 72 ml of a 1% itraconazole solution in methylene chloride were dripped into the reaction flask, under continuous stirring (6500 rpm) and heating (63° C.), for 220 minutes. With this operation completed, the reaction mixture was heated and stirred for yet 20 minutes in order to evaporate residual organic solvent.

The resulting dispersion comprising the itraconazole Solumer B was a clear yellow liquid with average particles size of 214.6 nm at 37° C. The viscosity was 130 cps (SpL2, 200 rpm). The content of itraconazole was 11.5 mg/ml as measured by HPLC (76.4% of the introduced amount of itraconazole).

### Example 4

### Preparation of Dispersions of Nanoparticles of Inclusion Complexes of Itraconazole Wrapped in Copolymer C (Solumer C)

For the preparation of the itraconazole Solumer C, 70 ml of Copolymer C solution prepared in Example 1(iii) was placed into a reaction flask. 85 ml of a 1% itraconazole solution in methylene chloride were dripped into the reaction flask, under continuous stirring (6500 rpm) and heating (65° C.), for 480 minutes. With this operation completed, the reaction mixture was heated and stirred for yet 30 minutes in order to evaporate residual organic solvent.

The resulting dispersion comprising the itraconazole Solumer C was a clear yellow liquid with average particles size of 146 nm at 37° C. The viscosity was 70 cps (SpL2, 200 rpm). The content of itraconazole as measured by HPLC was 8.8 mg/ml (62% of the initial theoretical amount), measured 5 month later.

### Example 5

### Preparation of Dispersions of Nanoparticles of Inclusion Complexes of Itraconazole Wrapped in Copolymer D (Solumer D)

For the preparation of the itraconazole Solumer D, 60 ml of Copolymer D solution prepared in Example 1(iv) were placed into a reaction flask. 82 ml of a 1% itraconazole solution in methylene chloride were dripped into the reaction flask, under continuous stirring (10500 rpm) and heating (63° C.), for 205 minutes. With this operation completed, the reaction mixture was heated and stirred for yet 20 minutes in order to evaporate residual organic solvent.

The resulting dispersion comprising the itraconazole Solumer D was a clear yellow liquid with average particles size of 234 nm at 37° C. The viscosity was 70 cps (SpL2, 200 rpm). The content of itraconazole as measured by HPLC was 14.3 mg/ml (95.8% of the initial theoretical amount), measured 1.5 months later.

### Example 6

### Preparation of Dispersions of Nanoparticles of Inclusion Complexes of Itraconazole Wrapped in Polymer E (Solumer E)

For the preparation of the itraconazole Solumer E, 200 ml of the Polymer E solution prepared in Example 1(v) were placed into a reaction flask. 200 ml of a 1% itraconazole solution in dichloromethane were dripped into the reaction flask, under continuous stirring (10500 rpm) and heating (62° C.), for 465 minutes. With this operation completed, the reaction mixture was heated and stirred for yet another 5 minutes in order to evaporate residual organic solvent.

The resulting dispersion comprising the itraconazole Solumer E was a clear yellow liquid with average particles size of 80 nm at 37° C. The viscosity was 365 cps (SpL2, 60 rpm). The content of itraconazole as measured by HPLC was 9.68 mg/ml (96.8% of the initial theoretical amount), measured one month following preparation.

### Example 7

### Concurrent Solumerization and Polymerization—One Step Process for Preparation of Dispersions of Nanoparticles of Inclusion Complexes of Itraconazole Wrapped in Acrylic Acid-Butyl Acrylate Copolymer (Solumer E)

For this one-step process, 6 g of ammonium peroxodisulfate in 140 ml of distilled water were placed into a reaction flask. An organic phase consisting of 64 ml of acrylic acid, 6 ml of butyl acrylate and 100 ml of methyl acetate was prepared, and 50 ml of this solution were added dropwise into the reaction flask during 75 min, under continuous stirring (6500 rpm) and heating (85° C.). The rest of the organic phase (120 ml) with itraconazole (6 g) dissolved therein, was dripped for the next 2 hours under the same conditions. On completion of the dripping, the reaction mixture was heated (85° C.) and stirred (6500 rpm) for yet 1 hour.

The resulting solumer, i.e. dispersion comprising nanoparticles of itraconazole wrapped in acrylic acid-butyl acrylate copolymer, was an opalescent viscous liquid. Its viscosity was 210 cps (25° C., SpL2, 100 rpm).

### Example 8

### Concurrent Solumerization and Polymerization—One Step Process for Preparation of Dispersions of Nanoparticles of Inclusion Complexes of Tebuconazole Wrapped in Acrylic Acid-Butyl Acrylate Copolymer

For this one-step process, 6 g of ammonium peroxodisulfate in 140 ml of distilled water were placed into a reaction flask. An organic phase consisting of 64 ml of acrylic acid, 6 ml of butyl acrylate, and 100 ml of methyl acetate was prepared, and 50 ml of this solution were added dropwise into the reaction flask during 75 min, under continuous stirring (6500 rpm) and heating (85° C.). The rest of the organic phase (120 ml) with tebuconazole (6 g) dissolved therein, was dripped for next 2 hours at the same conditions. On completion of the dripping, the reaction mixture was heated (85° C.) and stirred (6500 rpm) for yet 1 hour.

The resulting solumer, i.e., dispersion comprising nanoparticles of tebuconazole wrapped in acrylic acid-butyl acrylate copolymer was an opalescent viscous liquid. It had particles size 466 nm at 37° C., and viscosity 125 cps (25° C., SpL2, 200 rpm).

### Example 9

### Additional Itraconazole Hydrophilic Inclusion Complexes

Additional inclusion complexes of itraconazole were prepared according to the method described in Example 2 or 3, in which itraconazole was dissolved in methyl acetate or dichloromethane and the copolymer had different proportions of acrylic acid and butyl acrylate. Table 1 below shows the properties of various such itraconazole hydrophilic inclusion complexes.

TABLE 1
Properties of itraconazole hydrophilic inclusion complexes

| | | HPLC | | |
|---|---|---|---|---|
| | | | | Particle |
| | | Drug | % of | Size |
| Exp | Polymer (name/%) | (mg/ml) | Initial | nm |
| IT-50 | 30% Co-polymer | 8 | 80.1 | 80-100 |
| | (acrylic acid 26.25% and butyl | | | |
| | acrylate 3.75%) | | | |
| IT-51 | 43.75% Co-polymer | 10 | 94.4 | 70-80 |
| | (acrylic acid 38.25% and butyl | | | |
| | acrylate 5.5%) | | | |
| IT-52 | 33.33% Co-polymer | 10 | 98.4 | 70-110 |
| | (acrylic acid 29.33% and butyl | | | |
| | acrylate 4%) | | | |
| IT-OS-38-17 | 30% Co-polymer | 11.5 | 76.4 | 215 |
| | (acrylic acid : butyl acrylate | | | |
| | 24:1) | | | |
| IT-OS-43-43 | 30% Co-polymer (acrylic acid: | 14.3 | 95.8 | 234 |
| | methacrylic acid : butyl | | | |
| | acrylate 26.4:1.8:1.8) | | | |

The results in Table 1 show that when the insoluble itraconazole is wrapped within an amphiphilic acrylate copolymer, the resulting inclusion complex is hydrophilic.

### Example 10

### Oral Absorption of Nanosized Water-Soluble Particles of Itraconazole

The oral absorption of itraconazole nanosized water-soluble particles of itraconazole inclusion complexes with a 30% copolymer of acrylic acid (26.25%) and butyl acrylate (#7.5%) (#IT-50, Table 1) was studied in a preclinical model involving rats and compared with oral absorption of itraconazole in compositions comprising itraconazole mixed by vortex with polyacrylic acid, which do not form nanoparticles, in order to assess the contribution of the physical form for enabling absorption.

Itraconazole (50 mg/kg) is administered to male Sprague-Dawley rats (groups of 5), 250-280 g, by a feeding tube. At fixed times of administration (between 1-24 hours), blood samples are collected, and sera are prepared for analysis. At the end of the study, all rats are sacrificed by an IP overdose of pental (100 mg/kg).

Drug concentrations in rat serum (0.1 ml) are determined by HPLC using a method essentially as described by Yoo et al. (2002) Arch Pharm Res 25:387-391. The samples and calibration curve are prepared as follows: samples are mixed with an equal volume of acetonitrile to obtain a total volume of 400 μl. KCl is added to the samples for protein precipitation, and itraconazole, in the subsequent supernatant, is applied to a Merck HPLC system. The itraconazole concentration is quantified by comparison with a calibration curve in the range from 20 to 1000 ng/mL, that is prepared using blank rat serum spiked with itraconazole. A plot of the concentrations (not shown) is used to determine the timing of the maximal concentration (Cₘₐₓ) and to assess the total absorption of the drug (as reflected by the area under the curve (AUC).

A summary of the main pharmacokinetic findings is presented in Table 2. These findings demonstrate that administration of nanosized water-soluble particles having the same amount of intraconazole doubles the elevated maximal blood concentrations (Cₘₐₓ) of both itraconazole and its active hydroxylated metabolite (hydroxyitraconazole) and the total amount of itraconazole absorbed is increased, as reflected by the areas under the curve (AUC) of both itraconazole and its active hydroxylated metabolite.

TABLE 2
Comparison of pharmacokinetic parameters of itraconazole as water-
soluble particles (IT-50) and as mechanical mixture (MIX) with polymer

| | Itraconazole | | OH-itraconazole | |
|---|---|---|---|---|
| | IT-50 | MIX | IT-50 | MIX |
| Cₘₐₓ | 0.46 | 0.22 | 0.72 | 0.38 |
| Tₘₐₓ | 4 | 4 | 4 | 4 |
| AUC | 6.9 | 5.8 | 13.3 | 9.5 |

1. A hydrophilic inclusion complex consisting essentially of nanosized particles of an active compound and an amphiphilic polymer which wraps said active compound such that non-valent bonds are formed between said active compound and said amphiphilic polymer in said inclusion complex, wherein said amphiphilic polymer is a homopolymer of acrylic acid or methacrylic acid or a copolymer of acrylic acid or methacrylic acid, or both.

2. The hydrophilic inclusion complex according to claim 1, wherein said amphiphilic polymer is a copolymer of acrylic acid or methacrylic acid with one or two comonomers or a copolymer of acrylic acid, methacrylic acid and a third comonomer, wherein said comonomer is selected from the group consisting of acrylamide, methacrylamide, an alkyl acrylate, an alkyl methacrylate, acrylonitrile, ethyleneimine, vinyl acetate, styrene, maleic anhydride and vinyl pyrrolidone.

3. The hydrophilic inclusion complex according to claim 2, wherein said amphiphilic polymer is a copolymer of acrylic acid or methacrylic acid, or both, with an alkyl acrylate or an alkyl methacrylate.

4. The hydrophilic inclusion complex according to claim 3, wherein said alkyl acrylate or alkyl methacrylate is a straight, branched or cyclic (C₁-C₁₂)alkyl acrylate or (C₁-C₁₂)alkyl methacrylate.

5. The hydrophilic inclusion complex according to claim 4, wherein said alkyl acrylate or alkyl methacrylate is methyl acrylate, ethyl acrylate, butyl acrylate, n-octyl acrylate, 2-ethylhexyl acrylate or cyclohexyl methacrylate.

6. The hydrophilic inclusion complex according to claim 3, wherein said alkyl acrylate or alkyl methacrylate is a straight, branched or cyclic (C₁-C₁₂)alkyl acrylate or (C₁-C₁₂)alkyl methacrylate substituted by a radical selected from the group consisting of OH, —CONH₂, —NH₂, —COOH, —SO₃H, and —PO₃H₂.

7. The hydrophilic inclusion complex according to claim 6, wherein said substituted alkyl acrylate or alkyl methacrylate is 2-hydroxyethyl acrylate, 2-hydroxyethyl methacrylate, or 2-hydroxypropyl methacrylate.

8. The hydrophilic inclusion complex according to claim 1, wherein said active compound is in amorphous form.

9. The hydrophilic inclusion complex according to claim 1, wherein said active compound is selected from the group consisting of pharmaceutical compounds, food additives, cosmetics, pesticides and pet foods.

10. The hydrophilic inclusion complex according to claim 9, wherein said active compound is selected from the group consisting of vitamins, antibiotics and hormones.

11. The hydrophilic inclusion complex according to claim 9, wherein said active compound is a pharmaceutical compound.

12. The hydrophilic inclusion complex according to claim 9, wherein said active compound is an azole compound.

13. The hydrophilic inclusion complex according to claim 12, wherein the azole compound is an imidazole or triazole compound for human or veterinary application or for use in the agriculture.

14. The hydrophilic inclusion complex according to claim 13, wherein the azole compound is an azole fungicide for human application selected from the group consisting of terconazole, itraconazole, fluconazole, clotrimazole, miconazole, econazole, ketoconazole, tioconazole, isoconazole, oxiconazole, and fenticonazole.

15. The hydrophilic inclusion complex according to claim 14, wherein the azole fungicide for human use is itraconazole.

16. The hydrophilic inclusion complex according to claim 15, wherein the itraconazole is wrapped within an amphiphilic polymer selected from the group consisting of an acrylic acid-butyl acrylate and an acrylic acid-2(hydroxyethyl)methacrylate copolymer.

17. The hydrophilic inclusion complex according to claim 13, wherein the azole compound is a nonsteroidal antiestrogen selected from the group consisting of letrozole, anastrozole, vorozole, and fadrozole.

18. The hydrophilic inclusion complex according to claim 13, wherein the azole compound is an azole fungicide useful in the agriculture selected from the group consisting of bitertanol, cyproconazole, difenoconazole, epoxiconazole, fluquinconazole, flusilazole, flutriafol, hexaconazole, metconazole, myclobutanil, penconazole, propiconazole, tebuconazole, triadimefon, triadimenol, and triticonazole, imazalil, prochloraz, and triflumizole.

19. The hydrophilic inclusion complex according to claim 18, wherein the azole fungicide useful in the agriculture is tebuconazole.

20. The hydrophilic inclusion complex according to claim 13, wherein the azole compound is a nonfungicidal azole for use in the agriculture selected from the group consisting of azocyclotin, paclobutrazole, carfentrazone, isazophos, and metazachlor.

21. A hydrophilic dispersion comprising nanoparticles of inclusion complexes of an active compound and an amphiphilic polymer which wraps the active compound such that non-valent bonds are formed between said active compound and said amphiphilic polymer in said inclusion complex, wherein said amphiphilic polymer is a homopolymer of acrylic acid or methacrylic acid or a copolymer of acrylic acid or methacrylic acid, or both.

22. The hydrophilic dispersion according to claim 21, wherein said amphiphilic polymer is a copolymer of acrylic acid or methacrylic acid with one or two comonomers or a copolymer of acrylic acid, methacrylic acid and a third comonomer, wherein said comonomer is selected from the group consisting of acrylamide, methacrylamide, an alkyl acrylate, an alkyl methacrylate, acrylonitrile, ethyleneimine, vinyl acetate, styrene, maleic anhydride and vinyl pyrrolidone.

23. The hydrophilic dispersion according to claim 22, wherein said amphiphilic polymer is a copolymer of acrylic acid or methacrylic acid, or both, with an alkyl acrylate or an alkyl methacrylate.

24. The hydrophilic dispersion according to claim 23, wherein said alkyl acrylate or alkyl methacrylate is a straight, branched or cyclic (C₁-C₈)alkyl acrylate or (C₁-C₈)alkyl methacrylate.

25. The hydrophilic dispersion according to claim 24, wherein said alkyl acrylate or alkyl methacrylate is butyl acrylate, octyl acrylate, 2-ethylhexyl acrylate or cyclohexylmethacrylate.

26. The hydrophilic dispersion according to claim 24, wherein said alkyl acrylate or alkyl methacrylate is a straight or branched (C₁-C₁₂)alkyl acrylate or (C₁-C₁₂)alkyl methacrylate substituted by a radical selected from the group consisting of OH, —CONH₂, —NH₂, —COOH, —SO₃H, and —PO₃H₂.

27. The hydrophilic dispersion according to claim 26, wherein said substituted alkyl acrylate or alkyl methacrylate is 2-hydroxyethyl acrylate, 2-hydroxyethyl methacrylate, or 2-hydroxypropyl methacrylate.

28. The hydrophilic dispersion according to claim 21, wherein said active compound is in amorphous form.

29. The hydrophilic dispersion according to claim 21, wherein said active compound is selected from the group consisting of pharmaceutical compounds, food additives, cosmetics, pesticides and pet foods.

30. The hydrophilic dispersion according to claim 21, wherein said active compound is selected from the group consisting of vitamins, antibiotics and hormones.

31. The hydrophilic dispersion according to claim 29, wherein said active compound is a pharmaceutical compound.

32. The hydrophilic dispersion according to claim 29, wherein said active compound is an azole compound.

33. The hydrophilic dispersion according to claim 32, wherein the azole compound is an imidazole or triazole compound for human or veterinary application or for use in the agriculture.

34. The hydrophilic dispersion according to claim 33, wherein the azole compound is an azole fungicide for human application selected from the group consisting of terconazole, itraconazole, fluconazole, clotrimazole, miconazole, econazole, ketoconazole, tioconazole, isoconazole, oxiconazole, and fenticonazole.

35. The hydrophilic dispersion according to claim 40, wherein the azole fungicide is itraconazole.

36. The hydrophilic dispersion according to claim 41, wherein the itraconazole is wrapped within an amphiphilic polymer selected from the group consisting of an acrylic acid-butyl acrylate and an acrylic acid-2(hydroxyethyl)methacrylate copolymer.

37. The hydrophilic dispersion according to claim 33, wherein the azole compound is a nonsteroidal antiestrogen selected from the group consisting of letrozole, anastrozole, vorozole, and fadrozole.

38. The hydrophilic dispersion according to claim 33, wherein the azole compound is an azole fungicide useful in the agriculture selected from the group consisting of bitertanol, cyproconazole, difenoconazole, epoxiconazole, fluquinconazole, flusilazole, flutriafol, hexaconazole, metconazole, myclobutanil, penconazole, propiconazole, tebuconazole, triadimefon, triadimenol, and triticonazole, imazalil, prochloraz, and triflumizole.

39. The hydrophilic dispersion according to claim 44, wherein the azole fungicide useful in the agriculture is tebuconazole.

40. The hydrophilic dispersion according to claim 33, wherein the azole compound is a nonfungicidal azole for use in the agriculture selected from the group consisting of azocyclotin, paclobutrazole, carfentrazone, isazophos, and metazachlor.

41. A stable composition comprising a dispersion according to claim 21 and a carrier.

42. A stable pharmaceutical composition according to claim 41 comprising said dispersion and a pharmaceutically acceptable carrier.

43. A stable pesticidal composition according to claim 41 comprising said dispersion and an agriculturally acceptable carrier.

44. A process for preparation of a hydrophilic dispersion comprising nanoparticles of inclusion complexes of an active compound and an amphiphilic polymer which wraps the active compound such that non-valent bonds are formed between said active compound and said amphiphilic polymer in said inclusion complex, wherein said amphiphilic polymer is a homopolymer of acrylic acid, or methacrylic acid or a copolymer of acrylic acid or methacrylic acid, or both, the process comprising the steps of:
(i) preparing the amphiphilic homopolymer or copolymer of acrylic acid and/or methacrylic acid by reaction of the monomer(s) in water;
(ii) preparing a molecular solution of the active compound in an organic solvent;
(iii) dripping the cold solution of the active compound (ii) into the heated water homopolymer or copolymer solution (i) and heating at a temperature 5 to 10° C. above the boiling point of the organic solvent, under constant mixing; and
(iv) removing the organic solvent,
thus obtaining the hydrophilic dispersion comprising nanoparticles of inclusion complexes of said active compound wrapped within said amphiphilic homopolymer of acrylic acid or methacrylic acid or copolymer of acrylic acid or methacrylic acid, or both.

45. A one-step process for preparation of a hydrophilic dispersion comprising nanoparticles of inclusion complexes of an active compound and an amphiphilic polymer which wraps the active compound such that non-valent bonds are formed between said active compound and said amphiphilic polymer and said active compound is in a non-crystalline state in said inclusion complex, wherein said amphiphilic polymer is a homopolymer of acrylic acid, or methacrylic acid or a copolymer of acrylic acid or methacrylic acid, or both, the process comprising the steps of:
(iii) preparing a solution of the monomers in an organic solvent;
(ii) preparing a molecular solution of the active compound in a portion of the organic solution of (i);
(iii) dripping the remaining portion of solution (i) into a heated aqueous medium containing a polymerization initiator;
(iv) dripping the solution (ii) of the active compound into the heated aqueous initiator medium with the added monomers solution (i) and heating at 70-90° C., under constant mixing; and
(v) removing the organic solvent,
thus obtaining the hydrophilic dispersion comprising nanoparticles of inclusion complexes of said active compound wrapped within said amphiphilic homopolymer of acrylic acid or methacrylic acid or copolymer of acrylic acid or methacrylic acid, or both.

46. A one-step process for preparation of a hydrophilic dispersion comprising nanoparticles of inclusion complexes of an active compound and an amphiphilic polymer which wraps the active compound such that non-valent bonds are formed between said active compound and said amphiphilic polymer in said inclusion complex, wherein said amphiphilic polymer is a homopolymer of acrylic acid or methacrylic acid or a copolymer of acrylic acid or methacrylic acid, or both, the process comprising the steps of:
(iv) providing acrylic acid, acrylic acid or a mixture of monomers in liquid form;
(ii) preparing a molecular solution of the active compound in a portion of the monomer or the monomer mixture of (i);
(iii) dripping the remaining portion of the monomer or the monomer mixture of (i) into a heated aqueous medium containing a polymerization initiator; and
(iv) dripping the solution (ii) of the active compound into the heated aqueous initiator medium with the added monomers solution of (iii) and heating at 70-90° C., under constant mixing;
thus obtaining the hydrophilic dispersion comprising nanoparticles of inclusion complexes of said active compound wrapped within said amphiphilic homopolymer of acrylic acid or methacrylic acid or copolymer of acrylic acid or methacrylic acid, or both.
